(19) United States
(12) Patent Application Publication
(10) Pub. No.: US 2012/0232277 A1
(43) Pub. Date: Sep. 13, 2012

(54) PROCESS FOR THE PREPARATION OF 1,2,4-OXADIAZOL-3-YL DERIVATIVES OF CARBOXYLIC ACID

(75) Inventors: Kirill A. Lukin, Vernon Hills, IL (US); Vimal Kishore, Mundelein, IL (US); Thomas D. Gordon, Medway, MA (US)

(73) Assignee: ABBOTT LABORATORIES, Abbott Park, IL (US)

(21) Appl. No.: 13/415,054

(22) Filed: Mar. 8, 2012

Related U.S. Application Data
(60) Provisional application No. 61/450,437, filed on Mar. 8, 2011.

(51) Int. Cl. C07D 413/04; C07C 255/54; C07D 271/06; C07C 251/48; C07D 213/78; C07C 231/12; C07C 253/30; C07C 249/08

(52) U.S. Cl. 546/269.4; 558/414; 560/35; 546/298; 564/220; 548/131; 562/440

(57) ABSTRACT

The present invention discloses processes for the preparation and isolation of [1,2,4]oxadiazo-3-yl derivatives of carboxylic acids. The derivatives are useful in the treatment of inflammatory diseases and conditions.

### CROSS REFERENCE TO RELATED APPLICATION

This application claims priority to U.S. Provisional Application Ser. No. 61/450,437 filed on Mar. 8, 2011, the contents of which are incorporated herein.

### BACKGROUND

Alkylcarboxylic acids possessing heterocyclic substituents are important intermediates for the preparation of a useful class of biologically-active molecules. In particular, these biologically active compounds include but are not limited to S1P1 agonists such as those described in WO 2008076356 A1 and other publications. S1P1 agonists are useful, e.g., in the treatment of inflammatory diseases and conditions, and in the treatment of other diseases and conditions.

### SUMMARY OF THE INVENTION

In a first embodiment the invention provides a process for the preparation of a compound of Formula 4

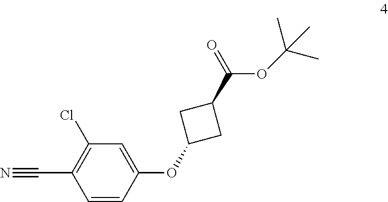

comprising the step of reacting compound of Formula 3

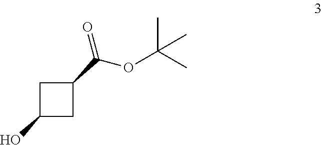

with a compound of Formula 5

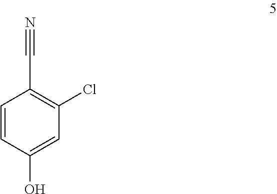

in the presence of activators such as TPP, DEAD or DIAD until the reaction is substantially complete to form a compound of Formula 4

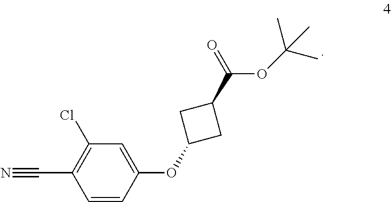

In a second embodiment the invention provides a process for the preparation of a compound of Formula 6

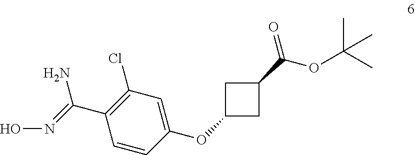

comprising the steps of reacting hydroxylamine with a compound of Formula 4

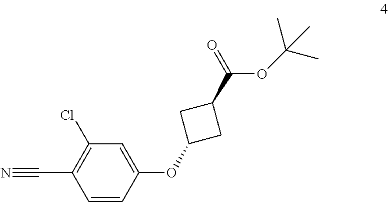

until the reaction is substantially complete, forming a compound of Formula 6

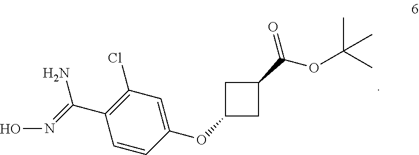

In a third embodiment the invention provides a process for the preparation of a compound of Formula 8

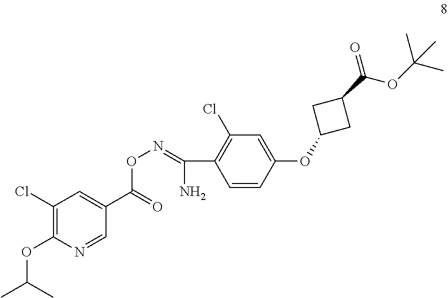

comprising the step of reacting compounds 6 and 7 in the presence of an activator such as carbonyldiimidazole, HATU or HOBT

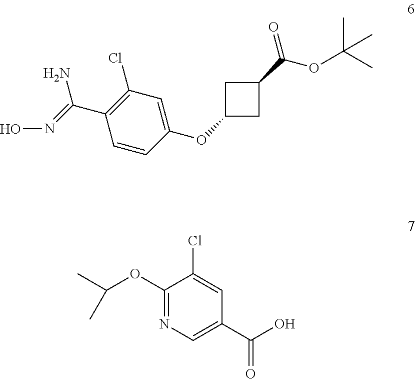

until the reaction is substantially complete to form a compound of Formula 8

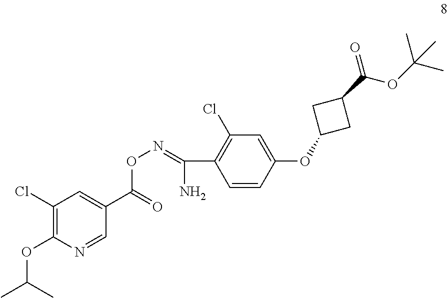

In a fourth embodiment the invention provides a process for the preparation of a compound of Formula 9

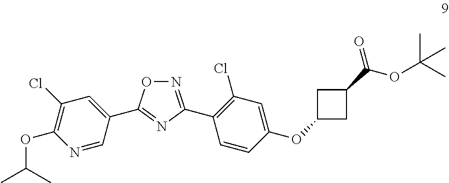

comprising of the cyclization of a compound of Formula 8

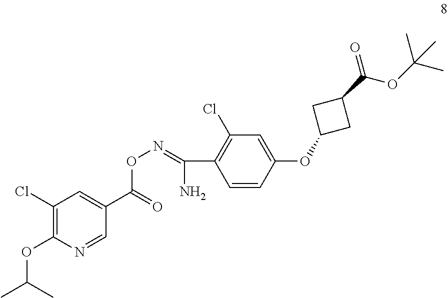

in the presence of a base such as tetrabutylammonium fluoride, diisopropylethylamine, DBU, or tetramethylguanidine until the reaction is substantially complete to form a compound of Formula 9

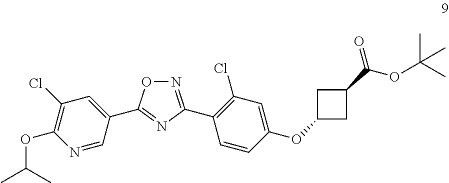

In a fifth embodiment the invention provides a process for the preparation of a compound of Formula 2

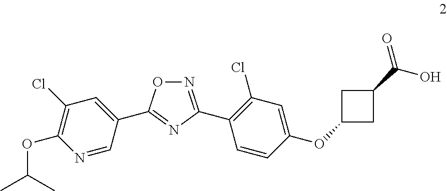

comprising the steps of reacting a compound of Formula 9

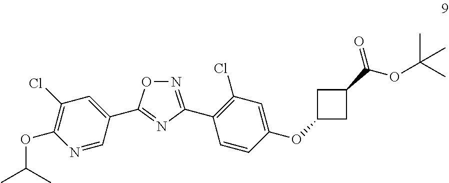

with triethylamine and trimethylsilyl triflate until the reaction is substantially complete to form a compound of Formula 2

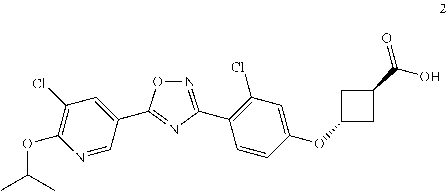

In a sixth embodiment the invention provides a process for the preparation of a compound of Formula 18

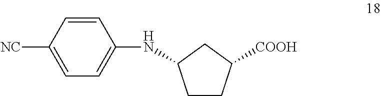

comprising the step of reacting the compounds of Formula 15 and Formula 16

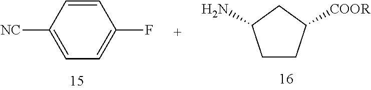

in the presence of a base such as potassium carbonate until the reaction is substantially complete to form a compound of Formula 18

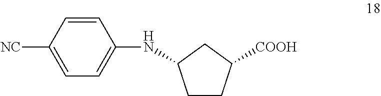

In a seventh embodiment the invention provides a process for the preparation of a compound of Formula 19

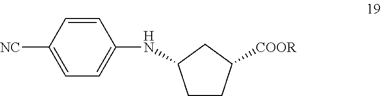

wherein R is alkyl, comprising the step of reacting a compound of Formula 18

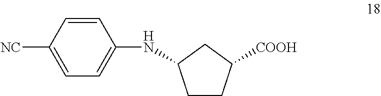

in a solution of hydrogen chloride gassed into an alcohol until the reaction is substantially complete to form a compound of Formula 19

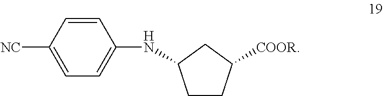

In an eighth embodiment the invention provides a process for the preparation of a compound of Formula 20

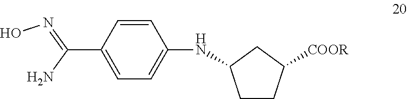

wherein R is alkyl, comprising the step of reacting a compound of Formula 19

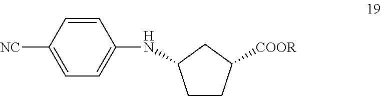

with hydroxylamine until the reaction is substantially complete to form a compound of Formula 20

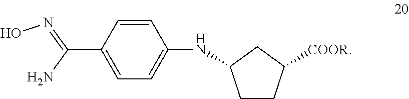

In a ninth embodiment the invention provides a process for the preparation of a compound of Formula 21

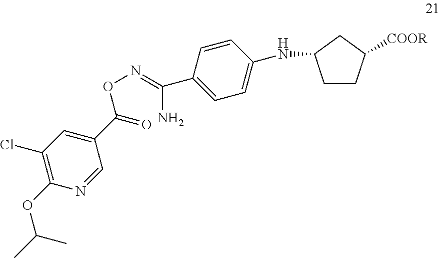

wherein R is alkyl, comprising the step of reacting a compound of Formula 20

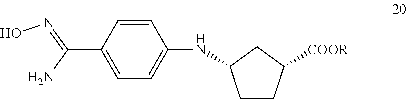

with DBU in THF until the reaction is substantially complete to form a compound of Formula 21

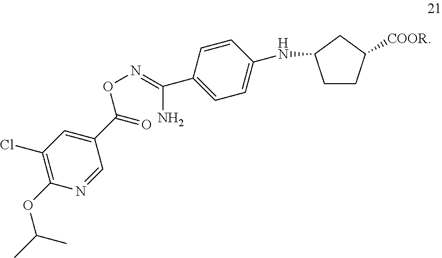

In a tenth embodiment the invention provides a process for the preparation of a compound of Formula 22

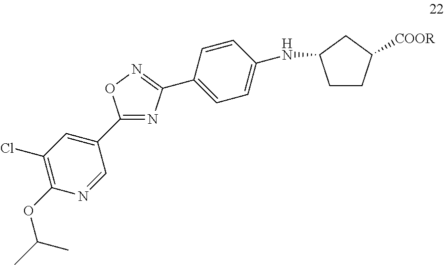

wherein R is alkyl, comprising the step of reacting a compound of Formula 21

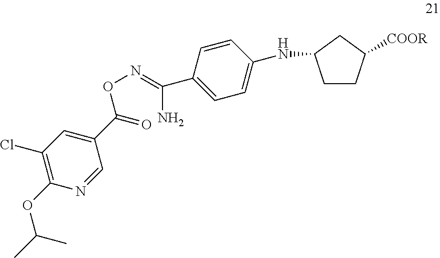

with DBU in THF until the reaction is substantially complete to form a compound of Formula 22

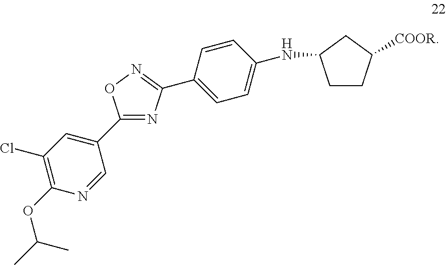

In an eleventh embodiment the invention provides a process for the preparation of a compound of Formula 23

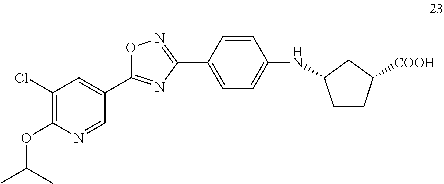

comprising the step of reacting sodium hydroxide and a compound of Formula 22

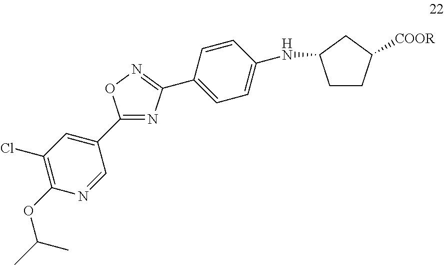

until the reaction is substantially complete to form a compound of Formula 23

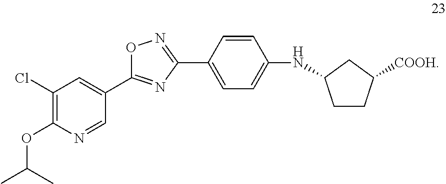

In a twelfth embodiment the invention provides a process for the preparation of a compound of Formula 26

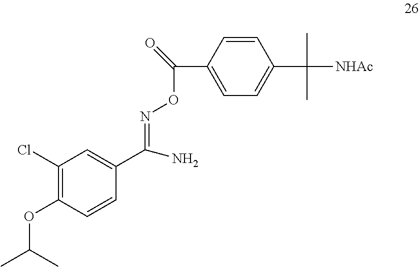

comprising the step of reacting compounds of Formula 24 and 25

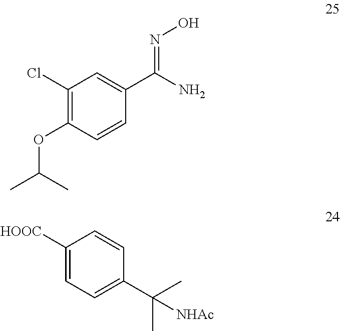

in the presence of an activator such as carbonyldiimidazole, HATU or HOBT until the reaction is substantially complete to form a compound of Formula 26

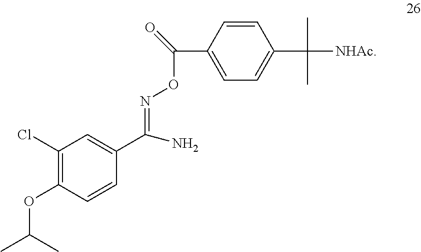

In a thirteenth embodiment the invention provides a process for the preparation of a compound of Formula 27

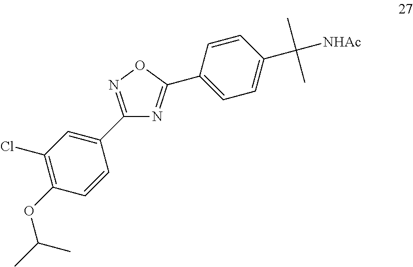

comprising of cyclization of a compound of Formula 26

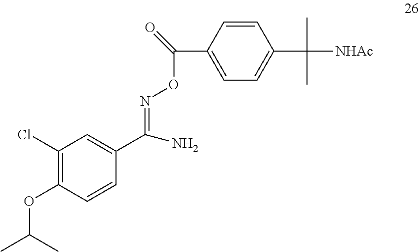

in the presence of a base such as until the reaction is substantially complete to form a compound of Formula 27

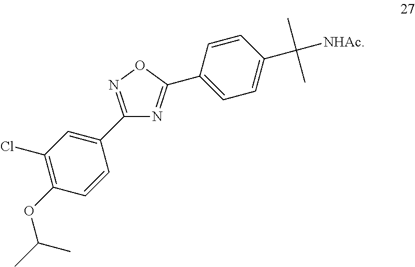

In a fourteenth embodiment the invention provides a process for the preparation of a compound of Formula 28

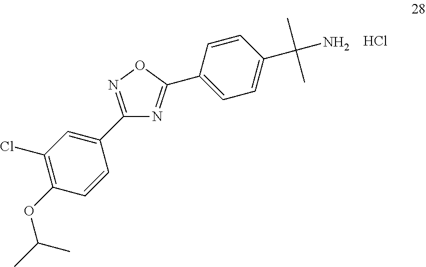

comprising the step of reacting a compound of Formula 27

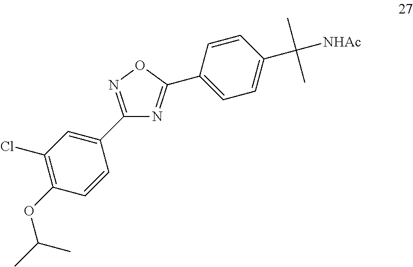

with pyridine and oxalyl chloride until the reaction is substantially complete to form a compound of Formula 28

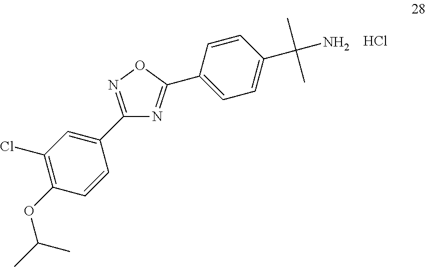

In a fifteenth embodiment the invention provides a process for the preparation of a compound of Formula 29

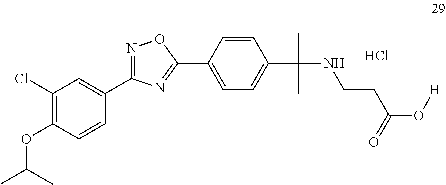

comprising the step of reacting a compound of Formula 28

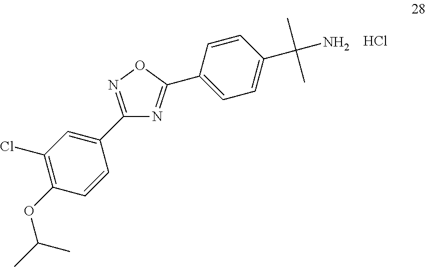

with alkyl acrylate until the reaction is substantially complete to form a compound of Formula 29

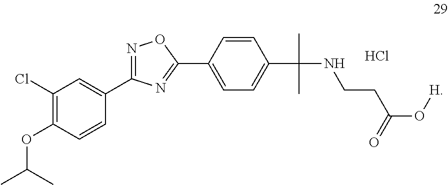

In a sixteenth embodiment the invention provides a process for the preparation of a compound of Formula 30

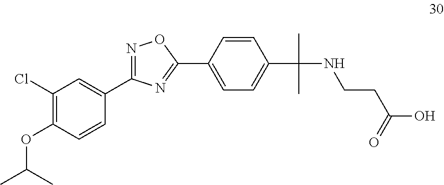

comprising the steps of reacting a compound of Formula 29

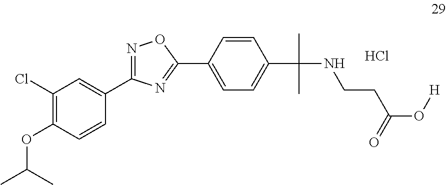

with sodium hydroxide until the reaction is substantially complete to form a compound of Formula 30

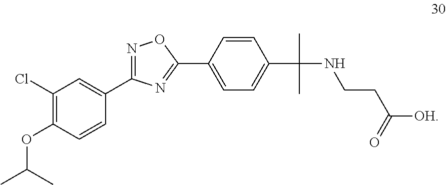

In a seventeenth embodiment the invention provides a process for the preparation of a compound of Formula 10

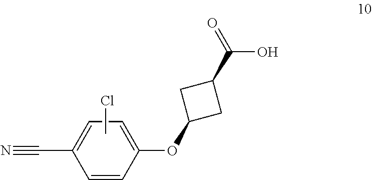

comprising the step of reacting the mixture of compounds of Formulas 3a and 3b

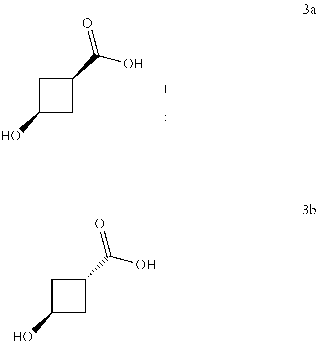

with a compound of Formula 5

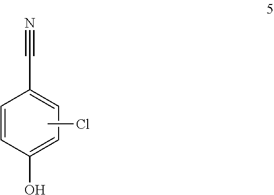

until the reaction is substantially complete to form a compound of Formula 10

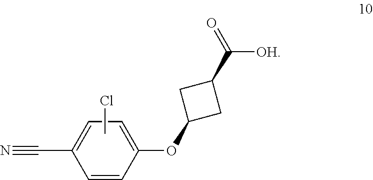

In an eighteenth embodiment the invention provides a process for the preparation of a compound of Formula 11

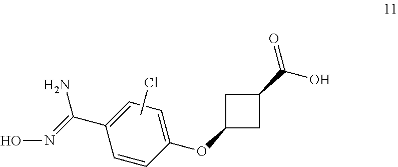

comprising the steps of reacting hydroxylamine with a compound of Formula 10

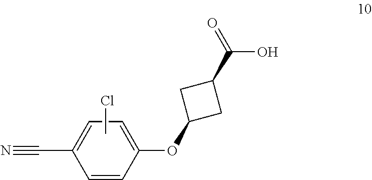

until the reaction is substantially complete, forming a compound of Formula 11

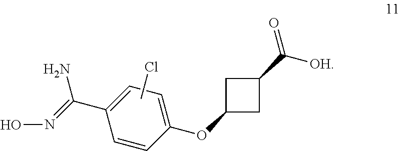

In a nineteenth embodiment the invention provides a process for the preparation of a compound of Formula 13

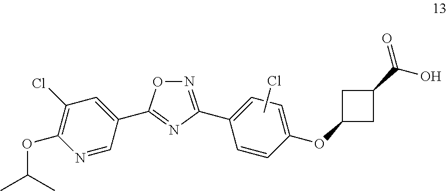

comprising the step of reacting carbonyldiimidazole with compounds 11 and 12

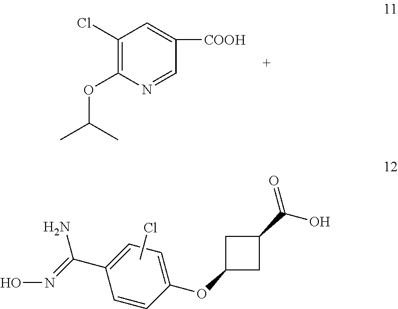

until the reaction is substantially complete to form a compound of Formula 13

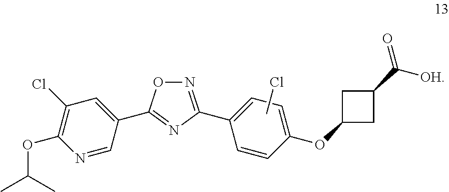

In a twentieth embodiment the invention provides a process for the preparation of a compound of Formula 14

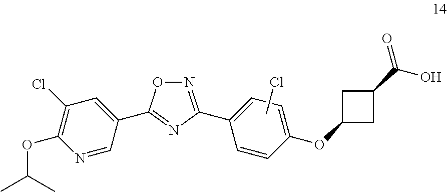

comprising the step of reacting a compound of Formula 13

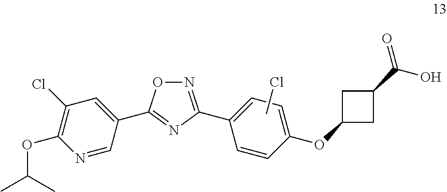

with sodium hydroxide until the reaction is substantially complete to form a compound of Formula 14

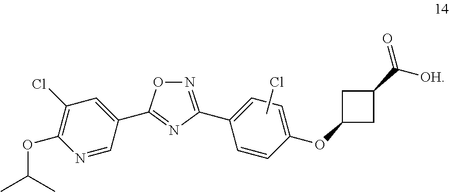

### DETAILED DESCRIPTION

### Abbreviations

ACN Acetonitrile
CAN Ceric ammonium nitrate
CDI Carbonyldiimidazole
DBU 1,8-Diazabicyclo[5.4.0]undec-7-ene
DCM Dichloromethane (methylene chloride)
DEAD Diethyl azodicarboxylate
DIEA N,N-Diisopropylethylamine
DMF N,N-Dimethylformamide
DMSO Dimethyl sulfoxide
EDC N-(3-Dimethylaminopropyl)-N′-ethylcarbodiimide hydrochloride
equiv Equivalent(s)
EtOH Ethanol
h Hour(s)
IPA Isopropyl alcohol
MTBE Methyl tert-butyl ether
Rₜ Retention Time
TEA Triethylamine
THF Tetrahydrofuran
TMSOTf Trimethylsilyltrifluoromethane sulfonate
TPP Triphenylphosphine
TPPO Triphenylphosphine oxide

### Analytical Methods

Analytical data is included within the procedures below. Unless otherwise stated, all ¹H data were collected at 500 MHz; chemical shifts are quoted in parts per million (ppm), mobile phase A was 0.1% phosphoric acid, mobile phase B was HPLC grade acetonitrile. HPLC data are referenced to the table of LC/MS and HPLC conditions using the method number provided in Table 1. High-pressure liquid chromatography (HPLC) analytical data are either detailed within the experimental or referenced to Table 1 of LC/MS and HPLC conditions.

TABLE 1
LC/MS and HPLC methods

| Method | Conditions |
|---|---|
| 1 | Column Eclipse XDB-C18 at 30° C.; detection at 205 nm; |
| | 0-5 min 100% A |
| | 5-15 min 0 to 100% B |
| | 15-20 min 100% B |
| 2 | Halo 4 min method: The gradient was 5-60% B in 1.5 min then 60-95% B to |
| | 2.5 min with a hold at 95% B for 1.2 min (1.3 mL/min flow rate). Mobile |
| | phase A was 10 mM NH₄OAc, mobile phase B was HPLC grade MeCN. The |
| | column used for the chromatography was a 4.6 × 50 mm MAC-MOD Halo C8 |
| | column (2.7 μm particles). Detection methods are diode array (DAD) and |
| | evaporative light scattering (ELSD) detection as well as positive/negative |
| | electrospray ionization. |
| 3 | Halo Purity QC method: The gradient was 5-60% B in 1.5 min then 60-95% |
| | B to 2.5 min with a hold at 95% B for 1.2 min (1.3 mL/min flow rate). |
| | Mobile phase A was 10 mM ammonium acetate, mobile phase B was HPLC |
| | grade acetonitrile. The column used for the chromatography was a 4.6 × 50 mm |
| | MAC-MOD Halo C8 column (2.7 μm particles). Detection methods are |
| | diode array (DAD) and evaporative light scattering (ELSD) detection as well |
| | as positive/negative electrospray ionization.) |
| 4 | The column used for the chromatography was an Altima C18 (25 cm) at 30° C.; |
| | detection at 205 nm; 0-15 min 5 to 100% B, 15-25 min 100% B. Mobile |
| | phase A was 0.1% perchloric acid, mobile phase B was HPLC grade ACN. |
| 5 | Column Zorbax Eclipse XDB C18 (15 cm) at 25° C.; detection at 205 nm; |
| | 0-18 min 20 to 95% B |
| | 18-22 min 95% B |

### General Synthetic Schemes

The general synthetic schemes that were utilized to construct the compounds disclosed in this application are described below.

The process for the synthesis and isolation of derivatives of carboxylic acids is shown in Schemes I-IV.

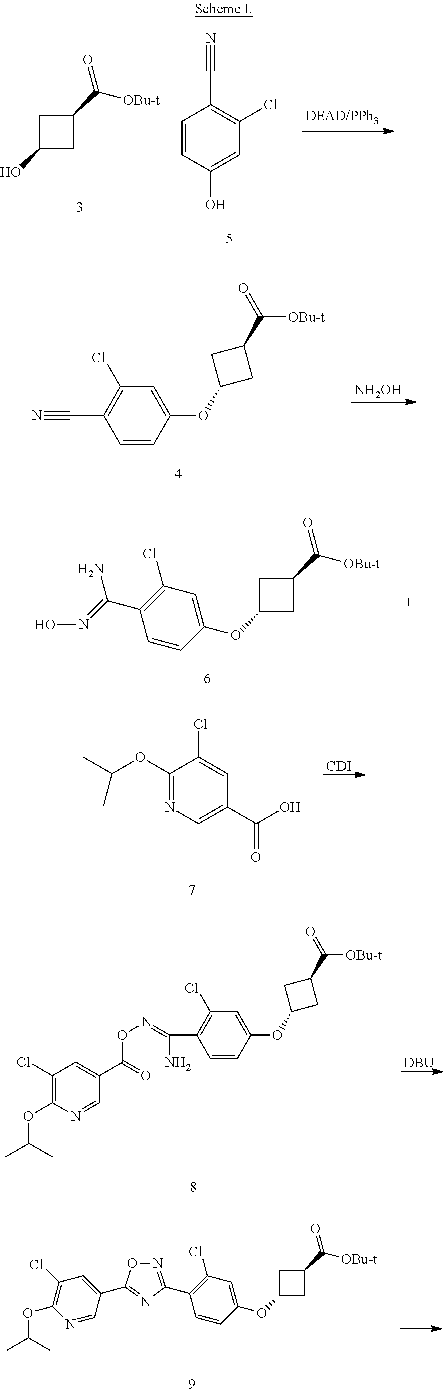

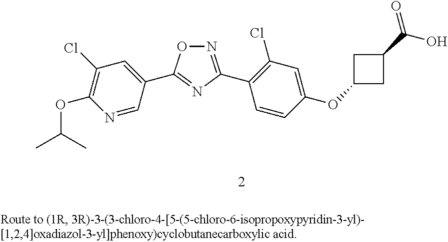

### Example #1

### (1R,3R)-3-{3-Chloro-4-[5-(5-chloro-6-isopropoxy-pyridin-3-yl)-[1,2,4]oxadiazol-3-yl]-phenoxy}cyclobutanecarboxylic acid

### Step 1. Preparation of (1R,3R)-tert-Butyl 3-(3-chloro-4-cyanophenoxy)cyclobutanecarboxylate

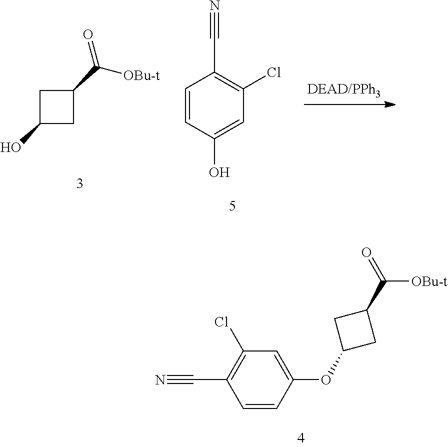

Alcohol 3 (˜50% solution in toluene, 1 equiv), phenol 5 (0.95 equiv) and triphenylphosphine (TPP, 1.2 equiv) in toluene (7 mL/g of 3) was cooled to about 0-5° C. and DEAD (40% solution in toluene, 1.0 equiv) was added while maintaining the internal temperature at no more than 25° C. The reaction mixture was then heated to about 25-30° C. for about 3-4 h. The mixture was cooled to about 20° C. and additional DEAD solution (0.2 equiv) was added while maintaining the internal temperature at about 30° C. The reaction was then continued at about 25-30° C. until completion (in-process sample: no more than 5% phenol vs. product by HPLC, typically about 10 h). Magnesium chloride (325 mesh, 2.5 equiv) was charged and the mixture was heated to about 60° C. The mixture was diluted with heptanes (10 mL/g of 3) and heating was continued until the TTPO concentration in solution was reduced to a target level (in-process test: no more than 5% peak area vs. toluene by HPLC). The mixture was cooled to ambient temperature and filtered to remove the solids. The filter cake was washed with toluene-heptanes (1:1, 10 mL/g of 3) and the combined filtrate containing the product was concentrated in vacuo and chased with IPA (10 mL/g of 3) to approximately 4.5 mL/g of 3 volume. The mixture was diluted with IPA to approximately 6 mL/g of 3 volume and cooled to no more than about 10° C. As product precipitation was observed the mixture was diluted with 2:1 IPA-water (15 mL/g of 3) to precipitate the remaining product. The product was filtered, washed with IPA-water and dried under vacuum at about 50° C. Typical yield of 4 was 75-80% PA.

¹H NMR (CDCl₃, δ, ppm) 1.48 (9H), 2.43 (2H), 2.68 (2H), 3.08 (1H), 4.90 (1H), 6.75 (1H), 6.88 (1H), 7.54 (1H). HPLC Method 1, Rₜ 9.1 min.

### Step 2. Preparation of (1R3R)-tert-Butyl 3-(3-chloro-4-((Z)—N′-hydroxycarbamimidoyl)phenoxy)cyclobutanecarboxylate

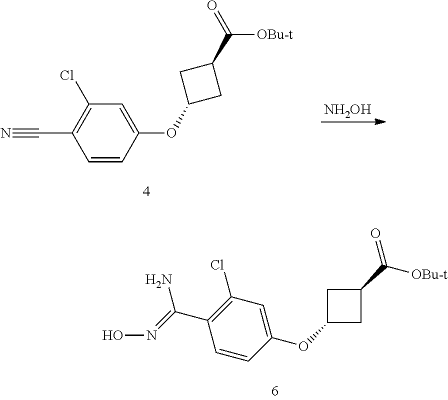

Hydroxylamine (50% in water, 4.8 equiv) was added to a solution of nitrile 4 in DMSO (6 mL/g of 4). The solution was slowly heated to about 50° C. and mixed at this temperature until the reaction was complete (in-process test: less than 1% starting material about 15 hours). The reaction mixture was cooled to about 20° C. and diluted with ethanol (3.5 mL/g of 4) and water (5.2 mL/g of 4) to precipitate the product. The mixture was agitated until the product concentration in the supernatant was reduced to less than 5 mg/mL). The product was filtered and washed with EtOH-water (1:1.5) (3.6 g/g of 4). The product was dried under vacuum at about 50° C. until residual water by Karl Fisher test—drying time was less than 0.5%. Typical yield of 6 was 90-93%.

¹H NMR (DMSOd₆, δ, ppm) 1.43 (9H), 2.32 (2H), 2.62 (2H), 3.05 (1H), 4.85 (1H), 5.72 (2H), 6.80 (1H), 6.87 (1H), 7.28 (1H), 9.37 (1H). HPLC Method 1, Rₜ 4.1 min.

### Step 3. Preparation of (1R,3R)-tert-Butyl 3-(3-chloro-4-((Z)—N′-(5-chloro-6-isopropoxynicotinoyloxy)carbamimidoyl)phenoxy)cyclobutanecarboxylate

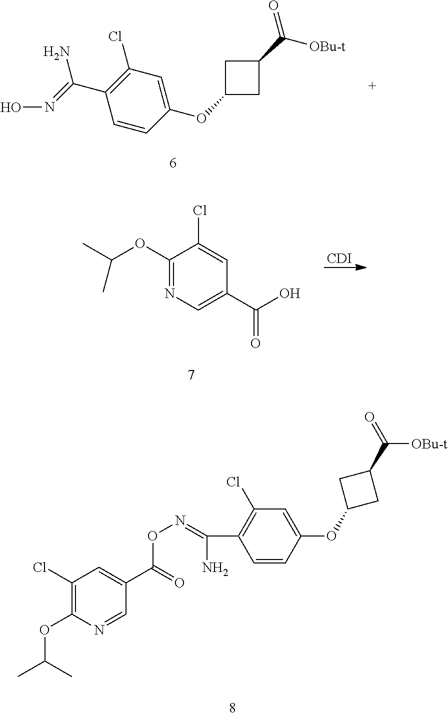

CDI (1.05 equiv) was dissolved in ACN (18 mL/g of CDI). The CDI solution in then transferred into a reactor containing nicotinic acid 7 (1.05 equiv) over about 5-10 min to control the carbon dioxide evolution. The vessel used to prepare the CDI solution was rinsed with ACN (2 mL/g CDI) and the rinse was transferred into the reaction mixture. After about 0.5 h the completeness of the imidazolide formation was checked (in-process sample was quenched into 1N DBU in methanol, followed by HPLC analysis; target less than 5% of 7 vs. its methyl ester). The solution of imidazolide was then transferred into a reactor containing imidoxime 6. Within a few minutes the product precipitated from the initially formed clear solution. After about 30 min the mixture was analyzed for the reaction completion (in-process test: less than 5% of 6 by HPLC). Then additional product was precipitated by water addition (9 mL/g of 6). The product was filtered, washed with 1:1 ACN-water (2 mL/g of 6) and dried under vacuum at no more than about 40° C. Typical yield of 8 was 92-97%.

¹H NMR (DMSOd₆, δ, ppm) 1.36 (6H), 1.43 (9H), 2.33 (2H), 2.63 (2H), 3.07 (1H), 4.90 (1H), 5.40 (1H), 5.72 (2H), 6.88 (1H), 6.97 (1H), 7.40 (1H), 8.59 (1H), 8.86 (1H). HPLC Method 1, Rₜ 10.0 min.

### Step 4. Preparation of (1R,3R)-tert-Butyl 3-(3-chloro-4-(5-(5-chloro-6-isopropoxypyridin-3-yl)-1,2,4-oxadiazol-3-yl)phenoxy)cyclobutanecarboxylate

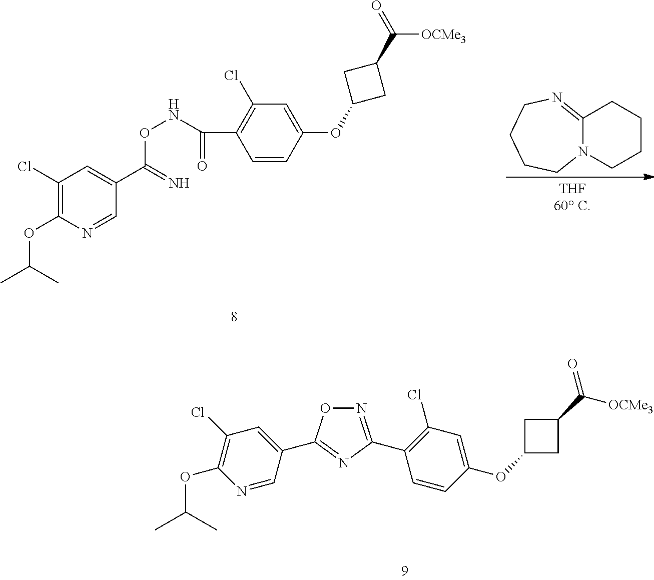

DBU (2 equiv) was charged to a solution of intermediate 8 in THF (5 mL/g of 8). The solution was heated to about 60° C. and mixed at this temperature until the reaction was complete (in-process test by HPLC<2% starting material, typically less than about 1 h). The reaction mixture was cooled to about 20° C. and the product was precipitated by the addition of water (5 mL/g of 8). The product was filtered, washed with 1:1 THF-water (2 mL/g of 8) and dried under vacuum at no more than about 60° C. Typical product yield was 95-99%.

¹H NMR (CDCl₃, δ, ppm) 1.44 (6H), 1.49 (9H), 2.45 (2H), 2.72 (2H), 3.09 (1H), 4.93 (1H), 5.47 (1H), 6.82 (1H), 6.92 (1H), 7.84 (1H), 8.36 (1H), 8.84 (1H). HPLC Method 1, Rₜ 12.9 min

### Step 5. Preparation of (1R,3R)-3-{3-Chloro-4-[5-(5-chloro-6-isopropoxy-pyridin-3-yl)-[1,2,4]oxadiazol-3-yl]-phenoxy}-cyclobutanecarboxylic acid

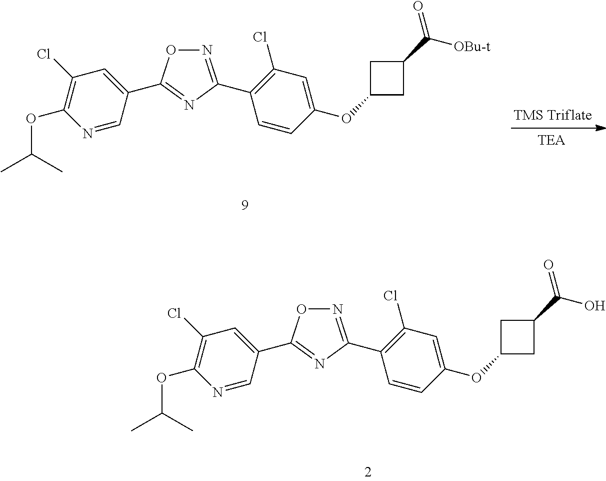

TEA (1.7 equiv) was added to a mixture of t-butyl ester (9, 1 equiv) in ethyl acetate (10 mL/g of 9 TMSOTf (1.6 equiv) was then added over about 30 min The reaction mixture was heated to about 70° C. and mixed at this temperature until the reaction was complete. (in-process test by HPLC: <0.5% starting material, about 2 h). The reaction mixture was cooled to about 20° C. and water (1 mL/g of 9) was added. The solvent was removed under vacuum (to about 3 mL volume/g of 9) to give a thick slurry. A mixture of ACN-water (1:2, 3.5 mL/g of 9) was added and the batch concentrated again to about 3 mL volume/g of 9. The residue was diluted with ACN-water (1:2, 17.5 mL/g of 9), and the pH of the mixture was adjusted to about 6. The slurry was mixed until product concentration in the supernatant drops to less than 0.5 mg/mL. The product was filtered and the cake was washed with ACN-water (1:2, 3.5 mL/g of 9). The product was dried under vacuum at about 60° C. until the residual water was reduced to less than 0.5% (Karl Fisher test, about 15 h). A typical yield of 2 was 95-98%.

¹H NMR (CDCl₃, δ, ppm) 1.44 (6H), 2.54 (2H), 2.83 (2H), 3.26 (1H), 4.97 (1H), 5.48 (1H), 5.72 (2H), 6.83 (1H), 6.96 (1H), 7.95 (1H), 8.35 (1H), 8.84 (1H). HPLC Method 1, Rₜ 10.3 min

### Scheme II. Route to (1R,3S)-3-(4-(5-(5-chloro-6-isopropoxypyridin-3-yl)-1,2,4-oxadiazol-3-yl)phenylamino)cyclopentanecarboxylic acid

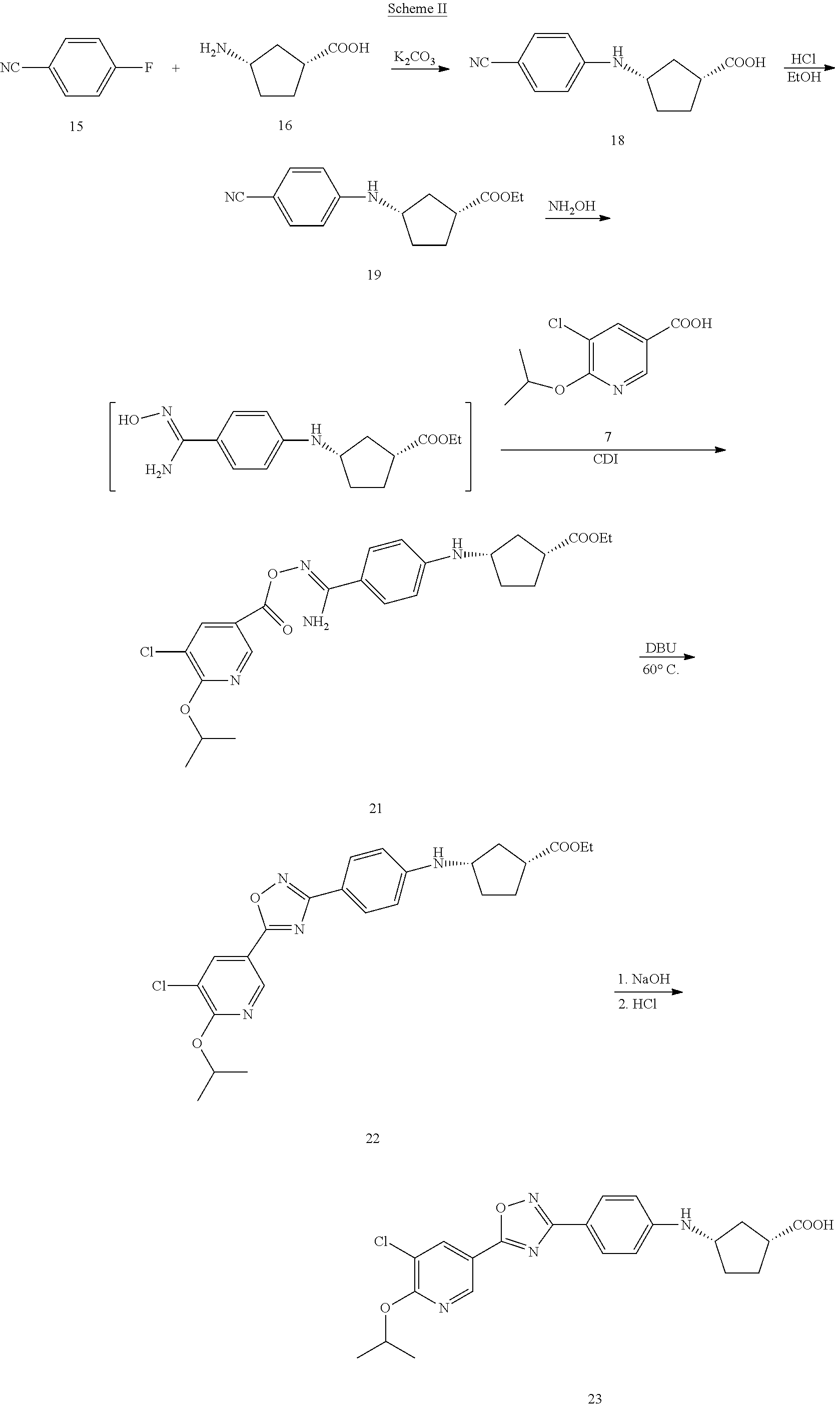

### Example #2

### (1R,3S)-3-(4-(5-(5-chloro-6-isopropoxypyridin-3-yl)-1,2,4-oxadiazol-3-yl)phenylamino)cyclopentanecarboxylic acid

### Step 1. Amino Acid Arylation

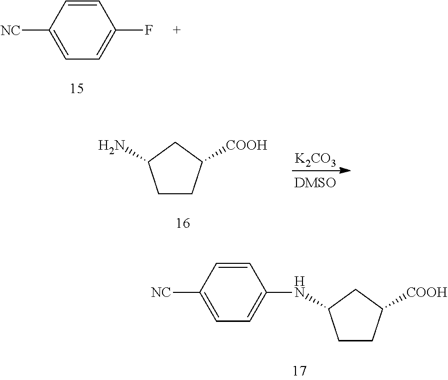

A mixture of amino acid 16 (1.0 equiv), fluorobenzonitrile 15 (1.0 equiv), and potassium carbonate (milled ˜300 mesh, 2.2 equiv) in DMSO-water (20:1; 6 mL/g of 15) was heated to about 105° C. with vigorous agitation until the reaction was complete (in process test by HPLC, typically about 16-20 h). The reaction mixture was cooled, diluted with MTBE (5 mL/g of 16) and water (10 mL/g of 16). The mixture was cooled to about 10° C. and concentrated HCl (0.7 g/g of 16) was added to dissolve solids. The aqueous layer was separated and the organic was extracted with 5% potassium carbonate solution. The product was precipitated via addition of concentrated HCl to the combined aqueous layer to achieve pH 4-6. The product slurry was then cooled to about 10° C. The product was filtered, washed with water (5 mL/g 12) and dried at 55° C. under vacuum. The yield for the amination step is typically 80%.

¹H NMR (DMSO-d₆, δ, ppm) 1.50 (1H), 1.60 (1H), 1.84 (2H), 1.96 (1H), 2.30 (1H), 2.74 (1H), 3.77 (1H), 6.60 (2H), 6.72 (1H), 7.41 (2H), 12.08 (1H). HPLC Method 4 Rₜ 12.0 min.

### Step 2. Esterification

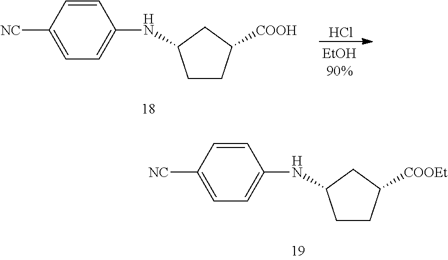

Hydrogen chloride (2.3 equiv) was gassed into ethanol (8 mL/g 18) while maintaining an internal temperature of not more than 20° C. The solution was then transferred to the acid 18 and the resulting solution was mixed until the reaction was complete (in process test by HPLC<1%, typically 2 h). The reaction mixture was cooled to about 10° C. and triethylamine was added to achieve pH 7-8. The product was then precipitated by water addition (10 mL/g of 18). The product was filtered, washed with water (5 mL/g 18) and dried at about 55° C. under vacuum. The potency adjusted yield for the esterification is typically 90-95%.

¹H NMR (DMSO-d₆, δ, ppm) 1.15 (3H), 1.51 (1H), 1.63 (1H), 1.86 (2H), 1.97 (1H), 2.30 (1H), 2.81 (1H), 3.77 (1H), 4.04 (2H), 6.60 (2H), 6.71 (1H), 7.41 (2H). HPLC Method 4, Rₜ 15.7 min.

### Step 3. Oximation

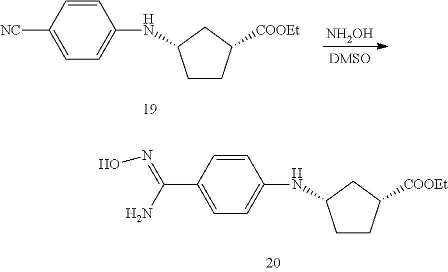

Hydroxylamine (50% in water, 4 equiv) was added to a solution of nitrile 19 in DMSO (5 mL/g of 19 while maintaining an internal temperature of not more than 20° C. The solution was slowly heated to about 50° C. and mixed until the reaction was complete (in process test by HPLC<5%, typically 6 h). The reaction mixture was cooled to about 20° C. and transferred into a mixture of water (10 mL/g of 19) and ethyl acetate (5 mL/g of 19). The organic layer was separated and the aqueous is extracted again with ethyl acetate (5 mL/g of 19). The combined organic layers were filtered through a ‘FilterAid’ cartridge to remove insoluble polymer. The solution was concentrated in vacuo and the solvent was switched to ACN. The ACN solution was concentrated to approximately 4 vol/g of 19. The resulting solution was directly used in the following coupling step. Typical assay yield for this step is 80-85%.

¹H NMR (DMSO-d₆, δ, ppm) 1.16 (3H), 1.51 (1H), 1.60 (1H), 1.86 (2H), 1.97 (1H), 2.30 (1H), 2.81 (1H), 3.74 (1H), 4.04 (2H), 5.51 (2H), 5.82 (1H), 6.50 (2H), 7.36 (2H), 9.17 (1H). HPLC Method 4, Rₜ 10.6 min.

### Step 4. Coupling

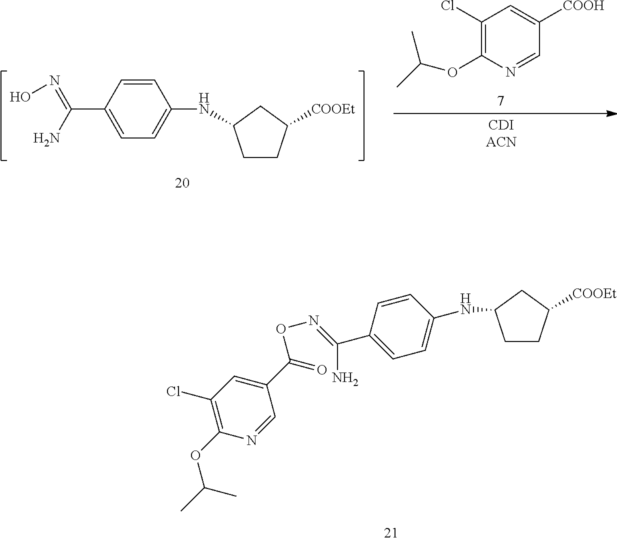

Nicotinic acid 7 (1.05 equiv) was slurried in ACN (2 mL/g of 7). In a separate vessel CDI (1.05 equiv) was slurried in CAN (10 mL/g of CDI). The CDI slurry was then transferred to the nicotinic acid slurry over about 10-15 min to control carbon dioxide evolution. The vessel used to prepare the CDI slurry was washed with ACN (2 mL/g CDI) and the wash was transferred into the reaction mixture. After about 0.5 h the in process, a sample was taken to check for the completeness of the imidazolide formation (quench into MeOH/DBU, followed by HPLC analysis, target less than 5% of the acid vs. methyl ester). The solution of imidoxime 20 was then added to the imidazolide solution over not less than 15 min resulting in the product precipitation. After about 30 min the mixture was analyzed for the reaction completion (<5% of 20 by HPLC) and additional product was precipitated by addition of water (9 mL/g of 20). The product was filtered, washed with water (2 mL/g of 20) and dried under vacuum at not more than 40° C. Typical product yield is 75-80% for two steps (3-4).

¹H NMR (DMSO-d₆, δ, ppm) 1.16 (3H), 1.36 (6H), 1.51 (1H), 1.60 (1H), 1.86 (2H), 1.96 (1H), 2.30 (1H), 2.82 (1H), 3.78 (1H), 4.04 (2H), 5.40 (1H), 6.10 (1H), 6.57 (2H), 6.72 (2H), 7.48 (2H), 8.54 (1H), 8.84 (1H). HPLC Method 4, Rₜ 19.0 min.

### Step 5. Oxadiazole Formation

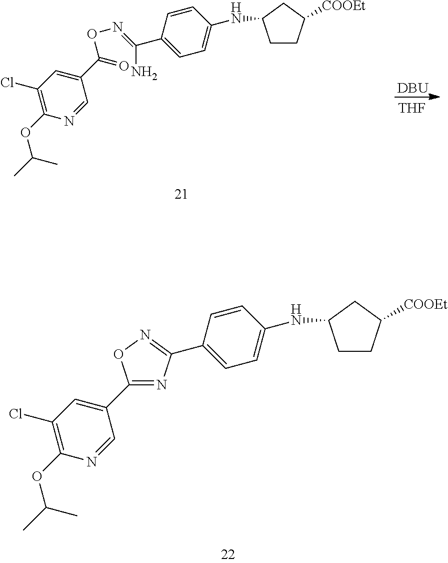

DBU (2 equiv) was charged to a slurry of intermediate 21 in THF (5 mL/g of 21). The solution was heated to about 60° C. and mixed at this temperature until the reaction was complete (in process test by HPLC<2%, about 2 h). The reaction mixture was cooled to about 20° C. and pH adjusted to 8-10 with concentrated HCl. The product was precipitated by addition of water (5 mL/g of 21). The product was filtered, washed with water (2 mL/g of 21) and dried under vacuum at not more than 60° C. Typical product yield is 90%.

¹H NMR (DMSO-d₆, δ, ppm) 1.16 (3H), 1.38 (6H), 1.51 (1H), 1.60 (1H), 1.88 (2H), 1.98 (1H), 2.33 (1H), 2.84 (1H), 3.81 (1H), 4.04 (2H), 5.43 (1H), 6.41 (1H), 6.68 (2H), 7.77 (2H), 8.47 (1H), 8.85 (1H). HPLC Method 4, Rₜ 22.1 min.

### Step 6. Deprotection

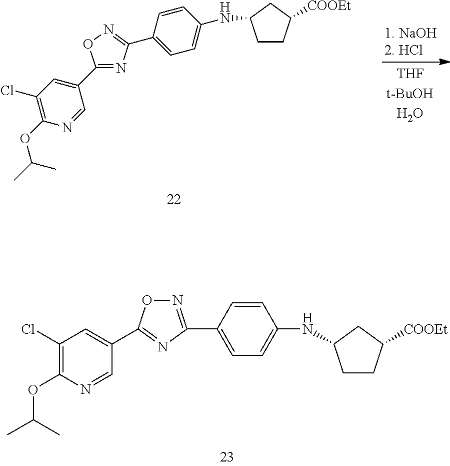

Sodium hydroxide (6.4% in water, 5 equiv) was charged to a slurry of intermediate 9 in THF (9 mL/g of 22) and t-butanol (3 mL/g of 22). The solution was mixed at about 20° C. until the reaction was complete (in process test by HPLC<0.5%, about 20-22 h). The reaction mixture was cooled to about 10° C. and pH adjusted to 8-10 with concentrated HCl. The mixture was concentrated in vacuo to about 6 mL/g of 22 volume, then chased with ethanol to about 6 mL/g of 22 volume. The mixture was diluted with ethanol (20 mL/g of 22 volume) and heated to about 45° C. The product was then precipitated by pH adjustment with 6N HCl to pH 5-6. Agitation was continued at about 50° C. for not less than 1 h. Then the internal temperature was slowly adjusted to about 15° C. The product was filtered off and washed with 1:1 ethanol/water, then with water.

The product was dried under vacuum initially at 55° C., then at 80° C. until the ethanol was reduced to less than 0.5 weight %. Typical product yield is 85-90%.

¹H NMR (DMSO-d₆, δ, ppm) 1.38 (6H), 1.52 (1H), 1.60 (1H), 1.86 (2H), 1.98 (1H), 2.31 (1H), 2.75 (1H), 3.76 (1H), 5.43 (1H), 6.45 (1H), 6.68 (2H), 7.76 (2H), 8.48 (1H), 8.85 (1H), 12.15 (1H). HPLC Method 4, Rₜ 20.0 min.

### Scheme III. Route to 3-(2-(4-(3-(3-chloro-4-isopropoxyphenyl)-1,2,4-oxadiazol-5-yl)propane-2-ylamino)propanoic acid

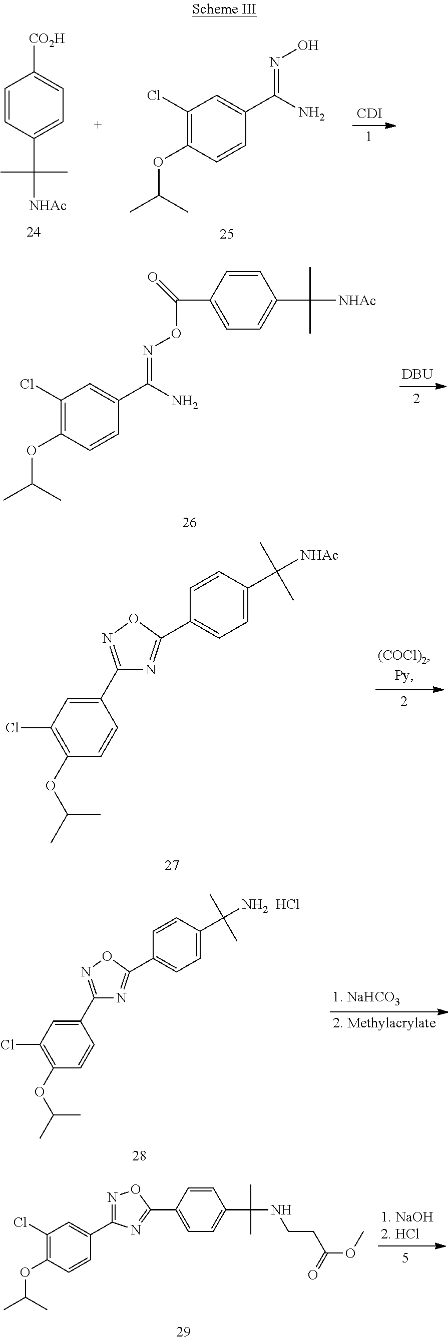

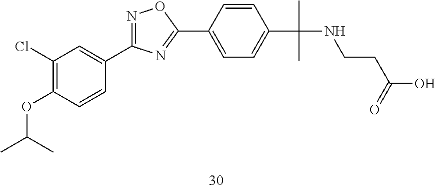

### Example #3

### 3-(2-(4-(3-(3-Chloro-4-isopropoxyphenyl)-1,2,4-oxadiazol-5-yl)propane-2-ylamino)propanoic acid

### Step 1. Coupling

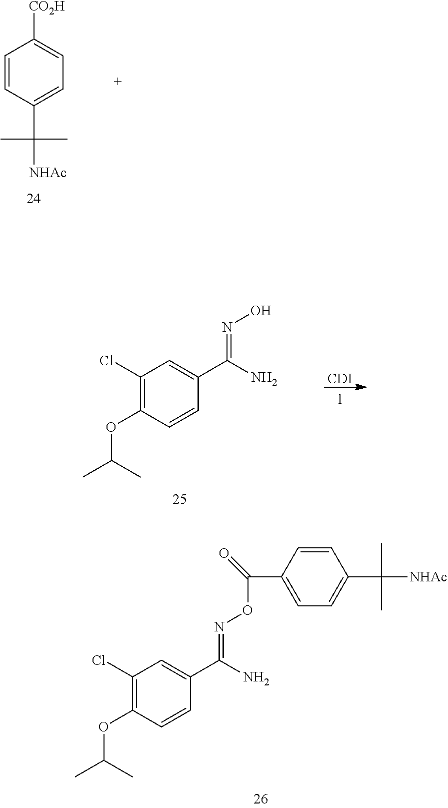

Benzoic acid 24 (1.03 equiv) was slurried in ACN (4 mL/g of 24). In a separate vessel CDI (1.1 equiv) is slurried in ACN (11 mL/g of CDI). The CDI slurry was then transferred to the benzoic acid slurry over 10-15 min to control carbon dioxide evolution. The vessel used to prepare the CDI slurry was washed with ACN (2 mL/g CDI) and the wash was transferred into the reaction mixture. After about 0.5 h the in process sample was taken to check for the completeness of the imidazolide formation (quench into MeOH/DBU, followed by HPLC analysis, target less than 5% of the acid vs. methyl ester). The solution of imidoxime 25 (1.0 equiv) in ACN (4 mL/g of 25) was then added to the imidazolide solution over not less than about 15 min. After about 1 h the mixture was analyzed for the reaction completion (<5% of 25 by HPLC) and concentrated in vacuo to about 10 mL/g of 25 volume, resulting in the product precipitation. The mixture was cooled to about 0° C. The product was filtered and dried under vacuum at not more than 45° C. Typical product yield was 95%.

¹H NMR (DMSO-d₆, δ, ppm) 1.31 (6H), 1.54 (6H), 1.83 (3H), 4.76 (1H), 6.90 (2H), 7.24 (1H), 7.43 (2H), 7.70 (1H), 7.81 (1H), 8.04 (2H), 8.17 (1H). HPLC Method 5, Rₜ 10.3 min.

### Step 2. Cyclization

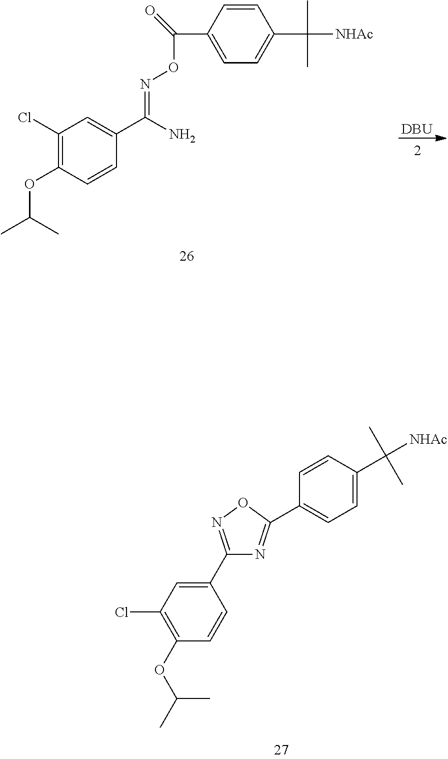

DBU (1.05 equiv) was charged to a slurry of intermediate 26 in dioxane (15 mL/g of 26). The solution was heated to about 100° C. and mixed at this temperature until the reaction was complete (in process test by HPLC<2%, typically 1 h). The reaction mixture was cooled to about 40° C. and concentrated in vacuo to 5 mL/g of 26 volume. The product was precipitated by addition of water (15 mL/g of 26). The product was filtered, washed with water and dried under vacuum at not more than about 60° C. Typical product yield was 85%.

¹H NMR (DMSO-d₆, δ, ppm) 1.33 (6H), 1.56 (6H), 1.84 (3H), 4.80 (1H), 7.36 (1H), 7.55 (2H), 7.98 (1H), 8.03 (1H), 8.07 (2H), 8.21 (1H). HPLC Method 5, Rₜ 15.5 min.

### Step 3. Deprotection

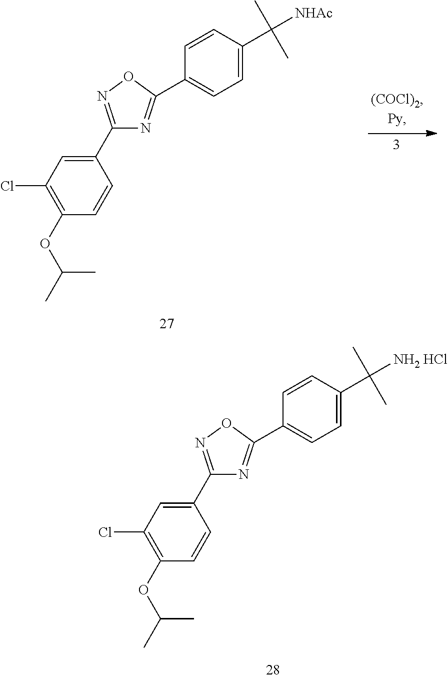

Pyridine (2 equiv) was charged to a solution of 27 in THF (17 mL/g of 27). The mixture was then cooled to about −5° C. and oxalyl chloride (1.5 equiv) was added slowly while maintaining not more than about 5° C. internal temperature. After mixing for about an additional 45 min at about 0° C. propylene glycol (2.5 equiv) was added slowly while maintaining not more than about 5° C. internal temperature. The reaction mixture was then heated to about 40-45° C. The heating was continued until the reaction is complete (in process test by HPLC). The mixture was quenched with 6N hydrochloric acid (2.5 equiv). After additional 1 h at about 40-45° C. the mixture was cooled to ambient temperature and diluted with water (5.5 mL/g of 27). The mixture was then concentrated in vacuo to remove THF and diluted with methyltetrahydrofurane (13 mL/g 27). The aqueous layer was separated from the resulting biphasic mixture and re-extracted with methyltetrahydrofurane (6 mL/g 27). Combined organic layers were concentrated in vacuo to about 4.5 mL/g of 27 volume, and the product was precipitated by the addition of ACN (10 mL/g of 27). The product was filtered, washed with ACN and dried under vacuum at not more than about 45° C. Typical product yield was 85%.

¹H NMR (DMSO-d₆, δ, ppm) 1.33 (6H), 1.69 (6H), 4.80 (1H), 7.37 (1H), 7.84 (2H), 7.98 (1H), 8.03 (1H), 8.21 (2H), 8.92 (3H). HPLC Rₜ 9.2 min.

### Step 4. Acrylate Addition

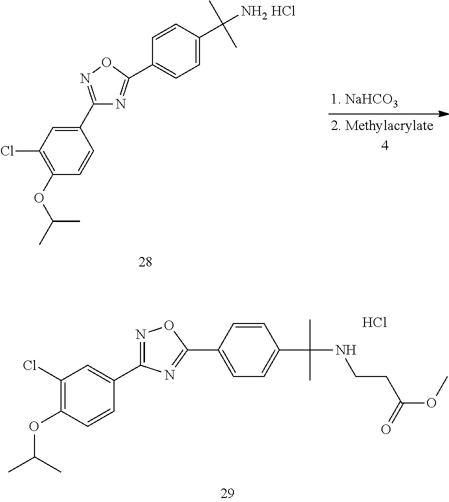

Amine HCl 28 was suspended in a mixture of Me-THF (15 mL/g 28) and 10% NaHCO₃ (7.5 mL/g 28). The mixing was continued until clear layers were formed. Layers were separated and the aqueous layer was extracted with more Me-THF (2×5 mL/g 28). The combined organic layers were washed with (2×5 mL/g 28 and then concentrated to near dryness in vacuo. The residue was dissolved in methanol (6 mL/g 28) and methyl acrylate (5 equiv) was added. The mixture was refluxed until the reaction was complete (in process test by HPLC<2%, typically 15-17 h). The mixture was then concentrated in vacuo and the residue was dissolved in ACN (17 mL/g of 28). The pH was adjusted to 2 with 4N hydrochloric acid. The resulting precipitate was filtered off and dried under vacuum. Typical product yield was 95%.

¹H NMR (DMSO-d₆, δ, ppm) 1.33 (6H), 1.78 (6H), 2.80 (4H), 3.58 (3H), 4.80 (1H), 7.37 (1H), 7.94 (2H), 7.98 (1H), 8.03 (1H), 8.23 (2H), 9.88 (2H). HPLC Rₜ 10.5 min.

### Step 5. Ester Hydrolysis

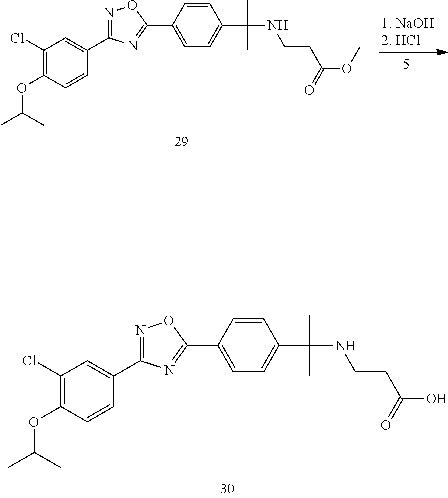

Sodium hydroxide (4 equiv, as 1N solution in water) was added to the mixture of methyl ester 29 in dioxane (6 mL/g 29). The mixture was heated to about 50° C. until the reaction is complete (in process test by HPLC<1%, typically 1 h). The pH was then adjusted to 6 with 1N hydrochloric acid resulting in the product precipitation. The product was filtered off and dried under vacuum at not more than about 55° C. Typical product yield was 90%.

¹H NMR (DMSO-d₆, δ, ppm) 1.33 (6H), 1.42 (6H), 2.28 (2H), 2.44 (2H), 4.80 (1H), 7.36 (1H), 7.72 (2H), 7.97 (1H), 8.02 (1H), 8.10 (2H). HPLC Rₜ 9.7 min.

### Scheme IV. Route to 3-(2-(4-(3-(3-chloro-4-isopropoxyphenyl)-1,2,4-oxadiazol-5-yl)propane-2-ylamino)propanoic acid

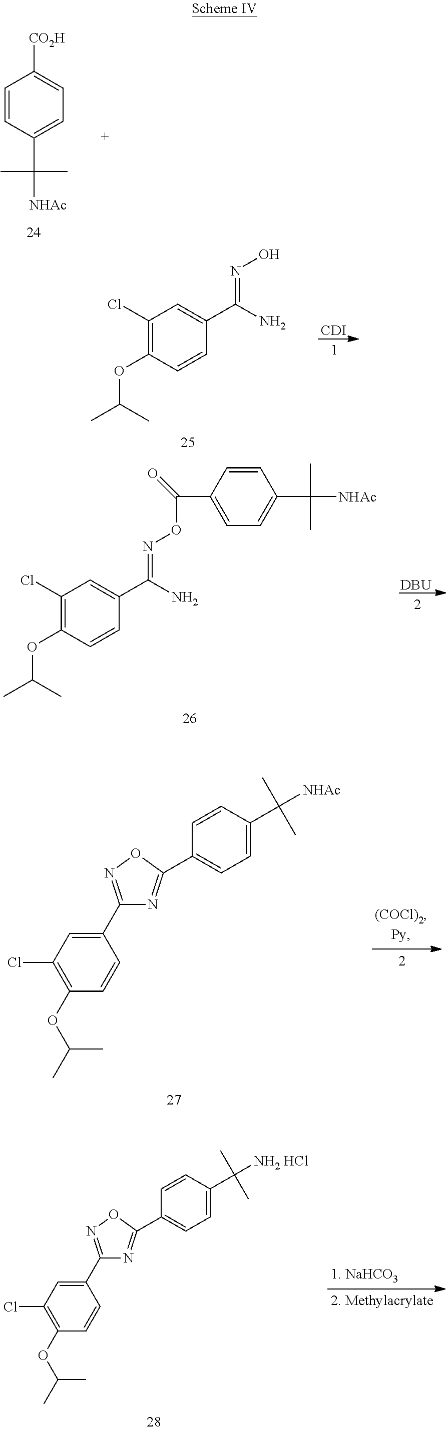

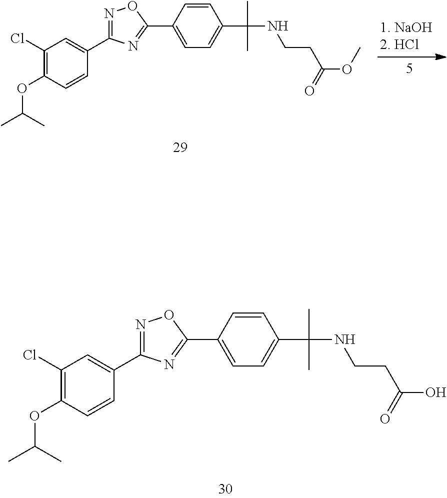

### Example #4

### 3-(2-(4-(3-(3-Chloro-4-isopropoxyphenyl)-1,2,4-oxadiazol-5-yl)propane-2-ylamino)propanoic acid

### Step 1. Coupling

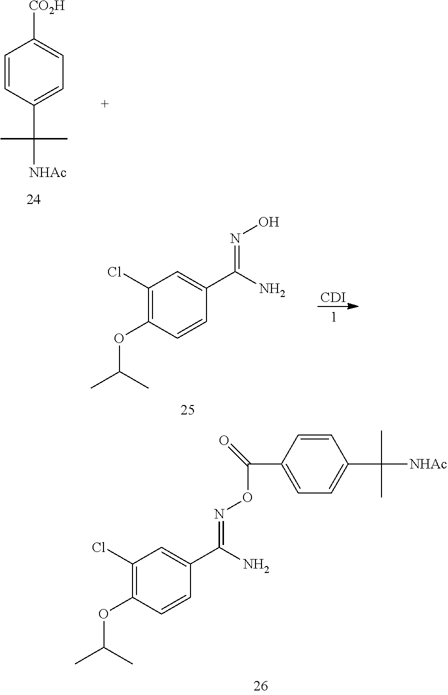

Benzoic acid 24 (1.03 equiv) was slurried in ACN (4 mL/g of 24). In a separate vessel CDI (1.1 equiv) is slurried in ACN (11 mL/g of CDI). The CDI slurry was then transferred to the benzoic acid slurry over 10-15 min to control carbon dioxide evolution. The vessel used to prepare the CDI slurry was washed with ACN (2 mL/g CDI) and the wash was transferred into the reaction mixture. After about 0.5 h the in process sample was taken to check for the completeness of the imidazolide formation (quench into MeOH/DBU, followed by HPLC analysis, target less than 5% of the acid vs. methyl ester). The solution of imidoxime 25 (1.0 equiv) in ACN (4 mL/g of 25) was then added to the imidazolide solution over not less than about 15 min. After about 1 h the mixture was analyzed for the reaction completion (<5% of 25 by HPLC) and concentrated in vacuo to about 10 mL/g of 25 volume, resulting in the product precipitation. The mixture was cooled to about 0° C. The product was filtered and dried under vacuum at not more than 45° C. Typical product yield was 95%.

¹H NMR (DMSO-d₆, δ, ppm) 1.31 (6H), 1.54 (6H), 1.83 (3H), 4.76 (1H), 6.90 (2H), 7.24 (1H), 7.43 (2H), 7.70 (1H), 7.81 (1H), 8.04 (2H), 8.17 (1H). HPLC Method 5, Rₜ 10.3 min.

### Step 2. Cyclization

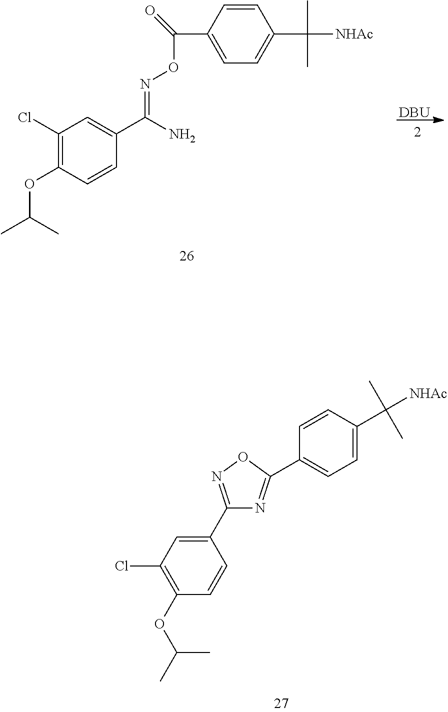

DBU (1.05 equiv) was charged to a slurry of intermediate 26 in dioxane (15 mL/g of 26). The solution was heated to about 100° C. and mixed at this temperature until the reaction was complete (in process test by HPLC<2%, typically 1 h). The reaction mixture was cooled to about 40° C. and concentrated in vacuo to 5 mL/g of 26 volume. The product was precipitated by addition of water (15 mL/g of 26). The product was filtered, washed with water and dried under vacuum at not more than about 60° C. Typical product yield was 85%.

¹H NMR (DMSO-d₆, δ, ppm) 1.33 (6H), 1.56 (6H), 1.84 (3H), 4.80 (1H), 7.36 (1H), 7.55 (2H), 7.98 (1H), 8.03 (1H), 8.07 (2H), 8.21 (1H). HPLC Method 5, Rₜ 15.5 min.

### Step 3. Deprotection

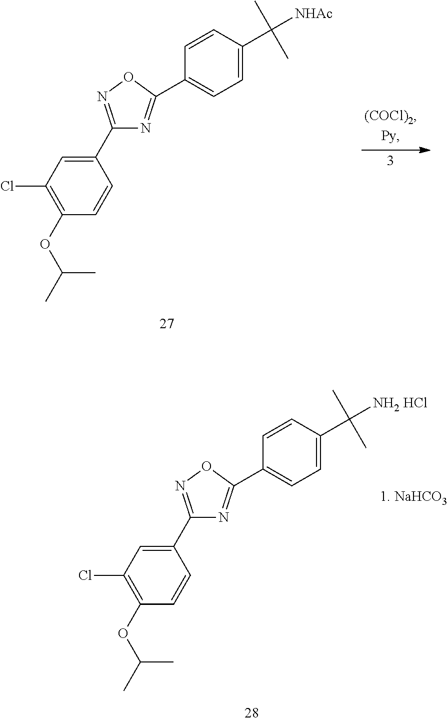

Pyridine (2 equiv) was charged to a solution of 27 in THF (17 mL/g of 27). The mixture was then cooled to about −5° C. and oxalyl chloride (1.5 equiv) was added slowly while maintaining not more than about 5° C. internal temperature. After mixing for about an additional 45 min at about 0° C. propylene glycol (2.5 equiv) was added slowly while maintaining not more than about 5° C. internal temperature. The reaction mixture was then heated to about 40-45° C. The heating was continued until the reaction is complete (in process test by HPLC). The mixture was quenched with 6N hydrochloric acid (2.5 equiv). After additional 1 h at about 40-45° C. the mixture was cooled to ambient temperature and diluted with water (5.5 mL/g of 27). The mixture was then concentrated in vacuo to remove THF and diluted with methyltetrahydrofurane (13 mL/g 27). The aqueous layer was separated from the resulting biphasic mixture and re-extracted with methyltetrahydrofurane (6 mL/g 27). Combined organic layers were concentrated in vacuo to about 4.5 mL/g of 27 volume, and the product was precipitated by the addition of ACN (10 mL/g of 27). The product was filtered, washed with ACN and dried under vacuum at not more than about 45° C. Typical product yield was 85%.

¹H NMR (DMSO-d₆, δ, ppm) 1.33 (6H), 1.69 (6H), 4.80 (1H), 7.37 (1H), 7.84 (2H), 7.98 (1H), 8.03 (1H), 8.21 (2H), 8.92 (3H). HPLC Rₜ 9.2 min.

### Step 4. Acrylate Addition

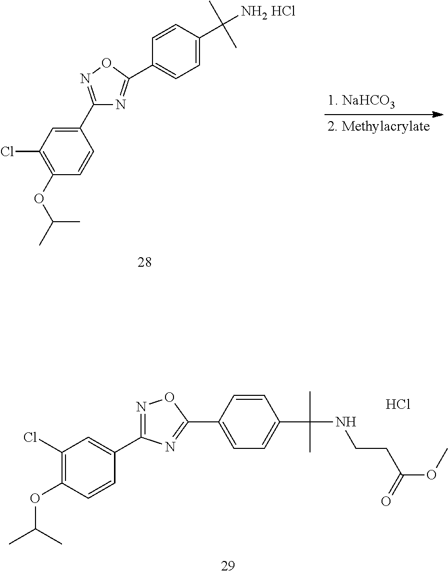

Amine HCl 28 was suspended in a mixture of Me-THF (15 mL/g 28) and 10% NaHCO₃ (7.5 mL/g 28). The mixing was continued until clear layers were formed. Layers were separated and the aqueous layer was extracted with more Me-THF (2×5 mL/g 28). The combined organic layers were washed with (2×5 mL/g 28 and then concentrated to near dryness in vacuo. The residue was dissolved in methanol (6 mL/g 28) and methyl acrylate (5 equiv) was added. The mixture was refluxed until the reaction was complete (in process test by HPLC<2%, typically 15-17 h). The mixture was then concentrated in vacuo and the residue was dissolved in ACN (17 mL/g of 28). The pH was adjusted to 2 with 4N hydrochloric acid. The resulting precipitate was filtered off and dried under vacuum. Typical product yield was 95%.

¹H NMR (DMSO-d₆, δ, ppm) 1.33 (6H), 1.78 (6H), 2.80 (4H), 3.58 (3H), 4.80 (1H), 7.37 (1H), 7.94 (2H), 7.98 (1H), 8.03 (1H), 8.23 (2H), 9.88 (2H). HPLC Rₜ 10.5 min.

### Step 5. Ester Hydrolysis

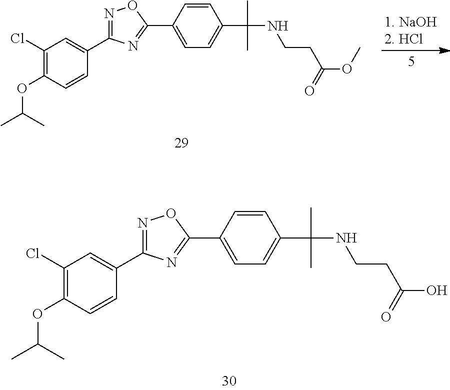

Sodium hydroxide (4 equiv, as 1N solution in water) was added to the mixture of methyl ester 29 in dioxane (6 mL/g 29). The mixture was heated to about 50° C. until the reaction is complete (in process test by HPLC<1%, typically 1 h). The pH was then adjusted to 6 with 1N hydrochloric acid resulting in the product precipitation. The product was filtered off and dried under vacuum at not more than about 55° C. Typical product yield was 90%.

¹H NMR (DMSO-d₆, δ, ppm) 1.33 (6H), 1.42 (6H), 2.28 (2H), 2.44 (2H), 4.80 (1H), 7.36 (1H), 7.72 (2H), 7.97 (1H), 8.02 (1H), 8.10 (2H). HPLC Rₜ 9.7 min.

The teachings of all references, including journal articles, patents and published patent applications, are incorporated herein by reference in their entirety.

What is claimed:

1. A process for the preparation of a compound of Formula 4 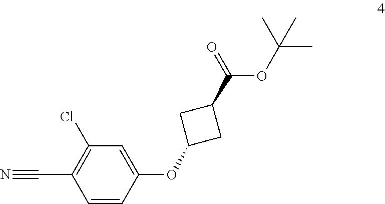
comprising the step of reacting compound of Formula 3 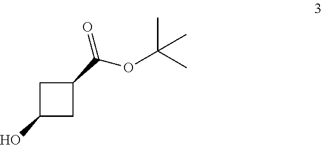
with a compound of Formula 5 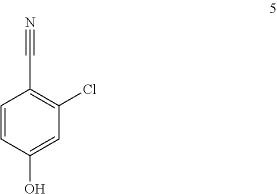
in the presence of activators such as TPP, DEAD or DIAD until the reaction is substantially complete to form a compound of Formula 4

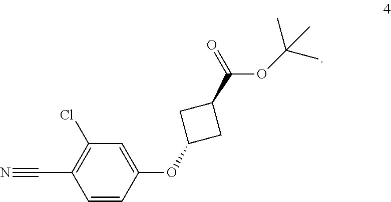

2. A process for the preparation of a compound of Formula 6 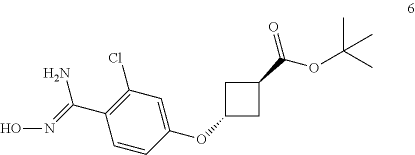
comprising the steps of reacting hydroxylamine with a compound of Formula 4 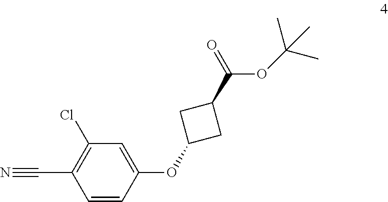
until the reaction is substantially complete, forming a compound of Formula 6

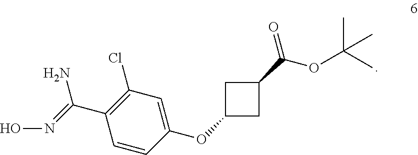

3. A process for the preparation of a compound of Formula 8 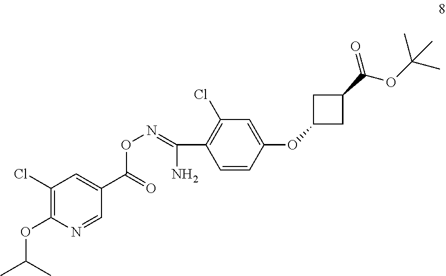
comprising the step of reacting compounds 6 and 7 in the presence of an activator such as carbonyldiimidazole, HATU or HOBT 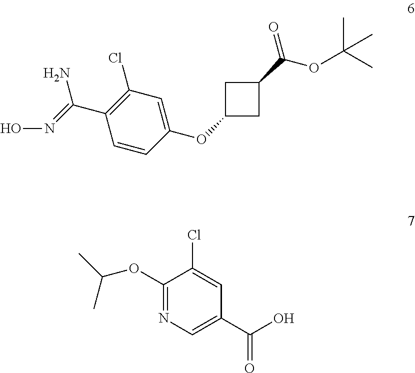
until the reaction is substantially complete to form a compound of Formula 8

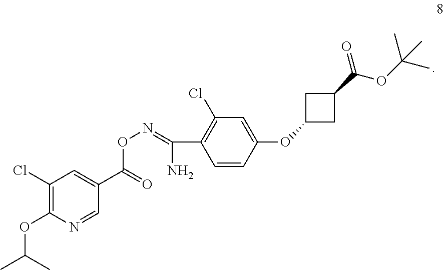

4. A process for the preparation of a compound of Formula 9 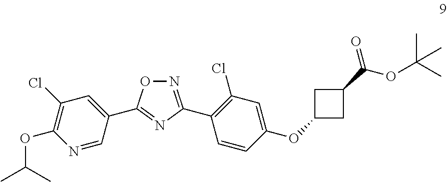
comprising of the cyclization of a compound of Formula 8 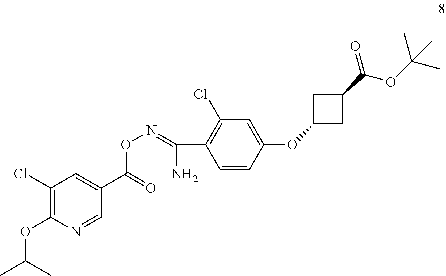
in the presence of a base such as tetrabutylammonium fluoride, diisopropylethylamine, DBU, or tetramethylguanidine until the reaction is substantially complete to form a compound of Formula 9

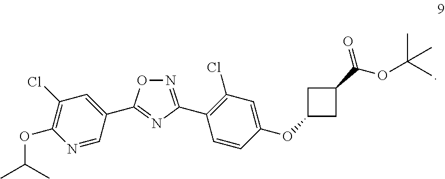

5. A process for the preparation of a compound of Formula 2 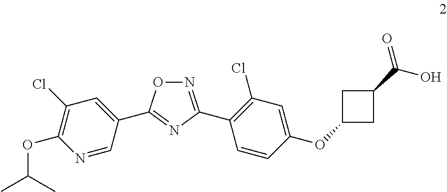
comprising the steps of reacting a compound of Formula 9 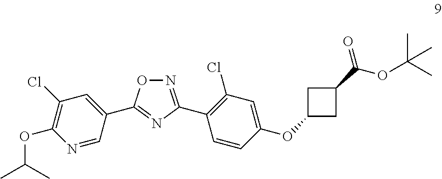
with triethylamine and trimethylsilyl triflate until the reaction is substantially complete to form a compound of Formula 2

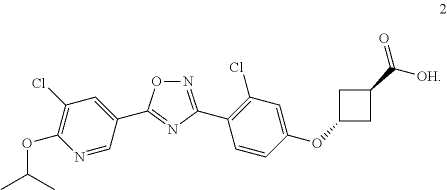

6. A process for the preparation of a compound of Formula 18 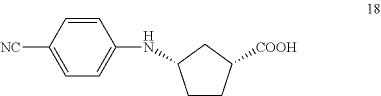
comprising the step of reacting the compounds of Formula 15 and Formula 16 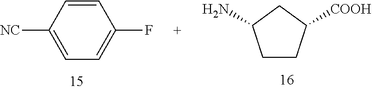
in the presence of a base such as potassium carbonate until the reaction is substantially complete to form a compound of Formula 18

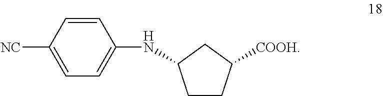

7. A process for the preparation of a compound of Formula 19 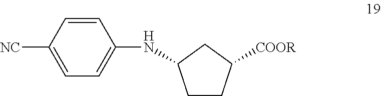
wherein R is alkyl, comprising the step of reacting a compound of Formula 18 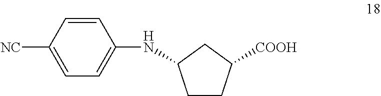
in a solution of hydrogen chloride gassed into an alcohol until the reaction is substantially complete to form a compound of Formula 19

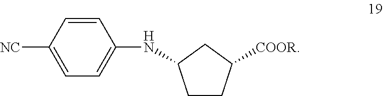

8. A process for the preparation of a compound of Formula 20 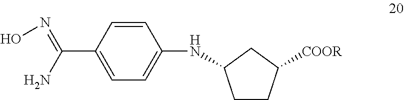
wherein R is alkyl, comprising the step of reacting a compound of Formula 19 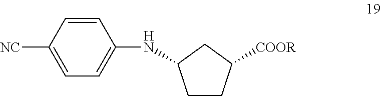
with hydroxylamine until the reaction is substantially complete to form a compound of Formula 20

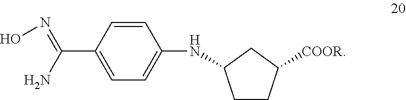

9. A process for the preparation of a compound of Formula 21 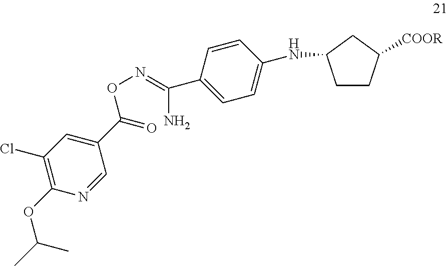
wherein R is alkyl, comprising the step of reacting a compound of Formula 20 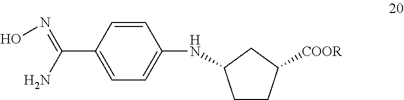
with DBU in THF until the reaction is substantially complete to form a compound of Formula 21 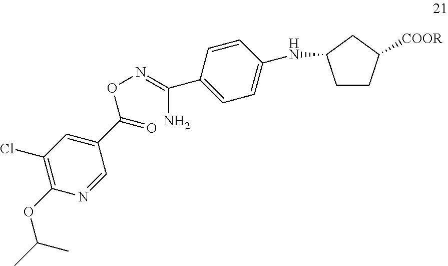
.

10. A process for the preparation of a compound of Formula 22 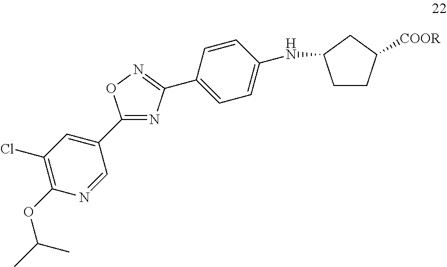
wherein R is alkyl, comprising the step of reacting a compound of Formula 21 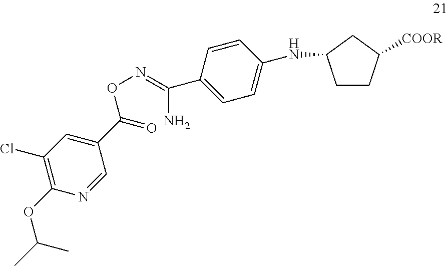
with DBU in THF until the reaction is substantially complete to form a compound of Formula 22

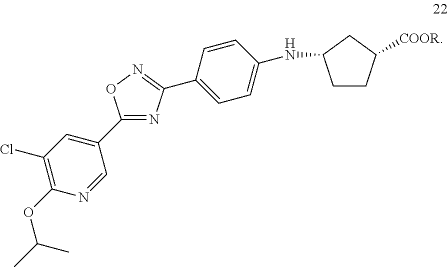

11. A process for the preparation of a compound of Formula 23 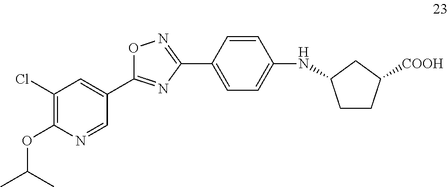
comprising the step of reacting sodium hydroxide and a compound of Formula 22 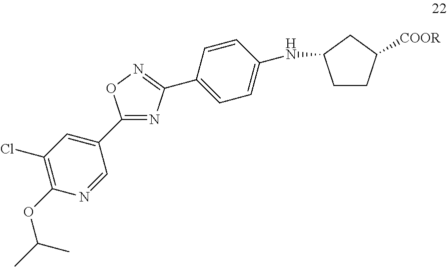
wherein R is alkyl, until the reaction is substantially complete to form a compound of Formula 23

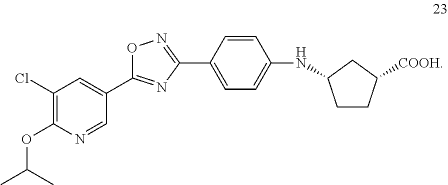

12. A process for the preparation of a compound of Formula 26 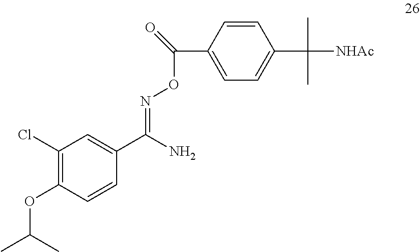
comprising the step of reacting compounds of Formula 24 and 25 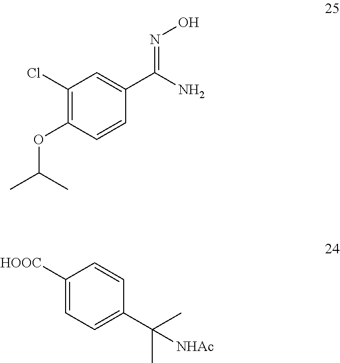
in the presence of an activator such as carbonyldiimidazole, HATU or HOBT until the reaction is substantially complete to form a compound of Formula 26

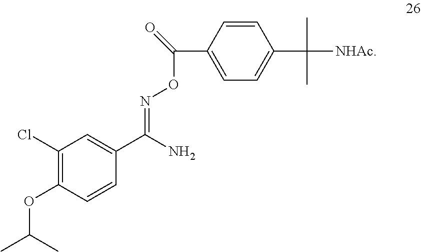

13. A process for the preparation of a compound of Formula 27 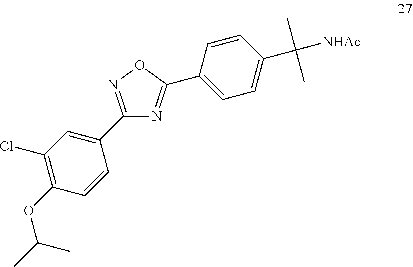
comprising of cyclization of a compound of Formula 26 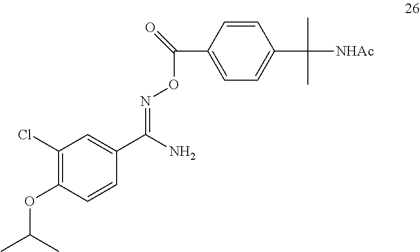
in the presence of a base such as until the reaction is substantially complete to form a compound of Formula 27

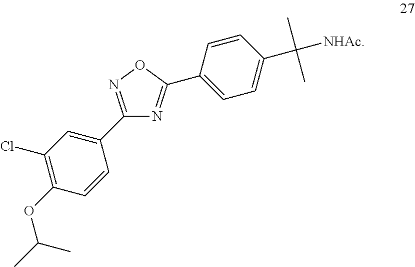

14. A process for the preparation of a compound of Formula 28 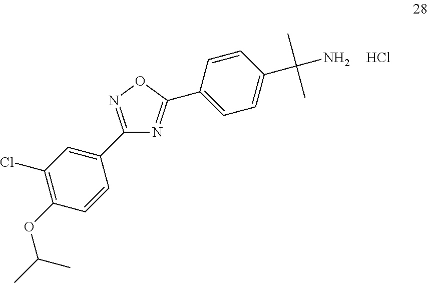
comprising the step of reacting a compound of Formula 27 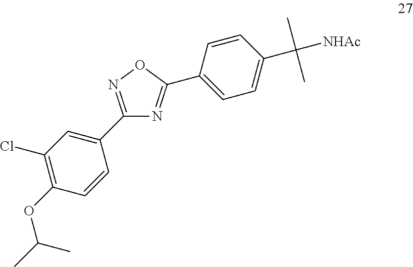
with pyridine and oxalyl chloride until the reaction is substantially complete to form a compound of Formula 28

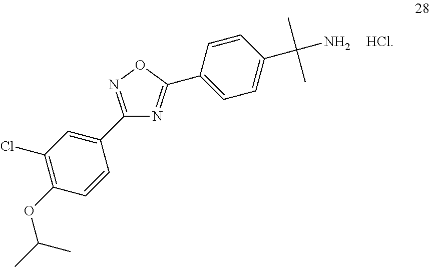

15. A process for the preparation of a compound of Formula 29 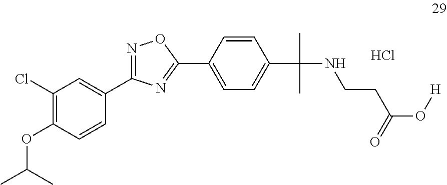
comprising the step of reacting a compound of Formula 28 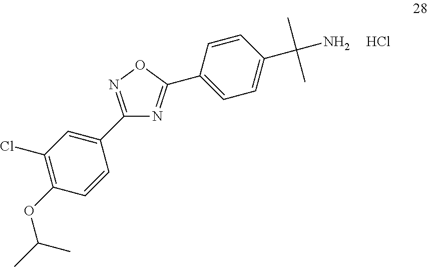
with alkyl acrylate until the reaction is substantially complete to form a compound of Formula 29

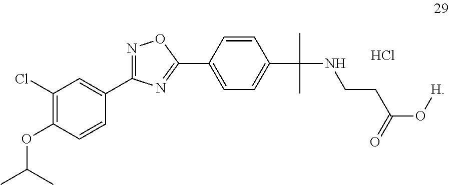

16. A process for the preparation of a compound of Formula 30 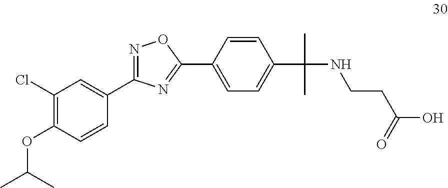
comprising the steps of reacting a compound of Formula 29 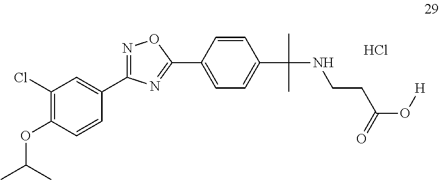
with sodium hydroxide until the reaction is substantially complete to form a compound of Formula 30

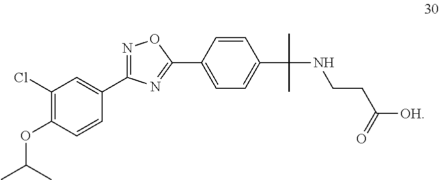

17. A process for the preparation of a compound of Formula 10 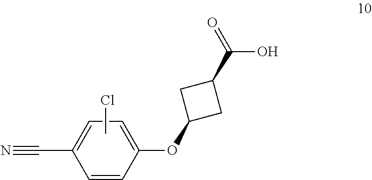
comprising the step of reacting the mixture of compounds of Formulas 3a and 3b 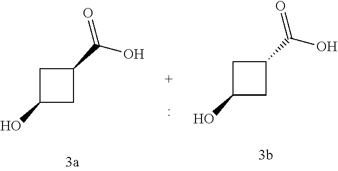
with a compound of Formula 5 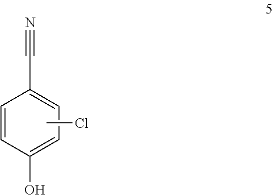
until the reaction is substantially complete to form a compound of Formula 10

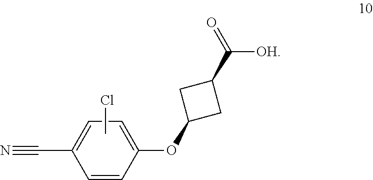

18. A process for the preparation of a compound of Formula 11 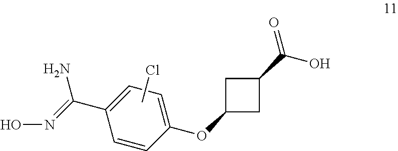
comprising the steps of reacting hydroxylamine with a compound of Formula 10 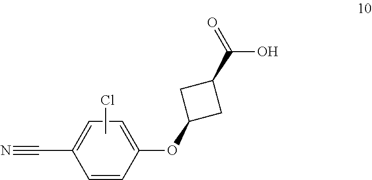
until the reaction is substantially complete, forming a compound of Formula 11

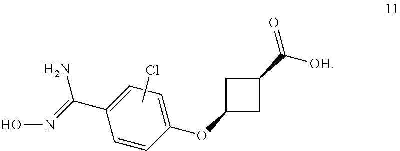

19. A process for the preparation of a compound of Formula 13 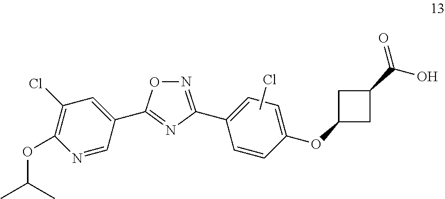
comprising the step of reacting carbonyldiimidazole with compounds 11 and 12 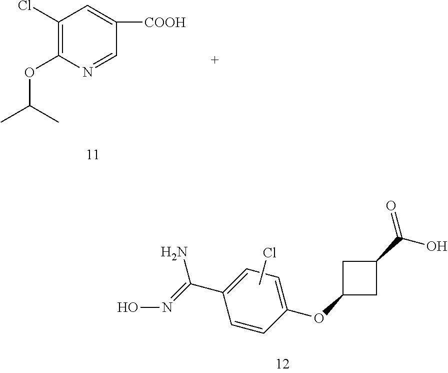
until the reaction is substantially complete to form a compound of Formula 13

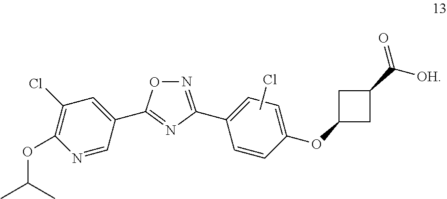

20. A process for the preparation of a compound of Formula 14 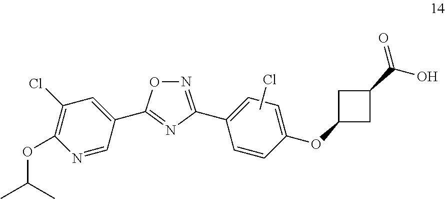
comprising the step of reacting a compound of Formula 13 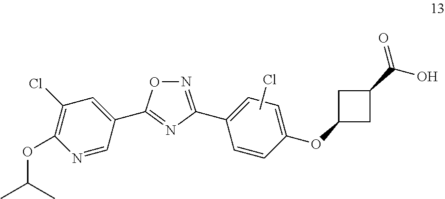
with sodium hydroxide until the reaction is substantially complete to form a compound of Formula 14 [chemical structure]
